(19) United States
(12) Patent Application Publication
(10) Pub. No.: US 2004/0022670 A1
(43) Pub. Date: Feb. 5, 2004

(54) System and method for decontaminating and/or sanitizing mail

(75) Inventors: Clifford A. Megerle, Thousand Oaks, CA (US); Duane Lee Lindner, San Ramon, CA (US)

Correspondence Address: PERKINS, SMITH & COHEN LLP, ONE BEACON STREET, 30TH FLOOR, BOSTON, MA, 02108, US

(73) Assignees: Lockheed Martin Corp., a Maryland corporation; Sandia National Laboratories

(21) Appl. No.: 10/282,370

(22) Filed: Oct. 29, 2002

Related U.S. Application Data
(60) Provisional application No. 60/330,808, filed on Oct. 31, 2001.

(51) Int. Cl. G05B 1/00; A01N 1/00

(52) U.S. Cl. 422/28; 422/32; 422/33; 422/37; 422/3

(57) ABSTRACT

A method and apparatus or system for de-contaminating and/or sanitizing mail in which a quantity of mail is placed into a confined chamber and the atmosphere in the chamber is evacuated to provide a sub-atmospheric pressure. The chamber is filled with an active gas that deactivates or degrades targeted bio-hazardous materials, and, after a pre-selected perio of time, the gas is evacuated from the confined chamber.

### CROSS-REFERENCE TO RELATED APPLICATIONS

The present application claims the priority of previously filed provisional application Serial No. 60/330,808 filed Oct. 31, 2001 for Method For De-Contaminating/Sanitizing Mail, the content of which is incorporated herein by reference.

### FIELD OF THE INVENTION

The present invention relates to a system and method for decontaminating and/or sanitizing mail, and, more particularly, to a system and method for decontaminating and/or sanitizing mail or similar articles involving the use of an active gas which degrades or deactivates bio-hazardous materials, particularly anthrax spores.

### BACKGROUND OF THE INVENTION

Some mail pieces have been known to contain dangerous biohazard materials, including particulates such as anthrax spores. In addition to seeking to expose the addressee to the biohazard material, the biohazard-containing piece also contaminates other mail pieces being handled in the mail transport system and also contaminates the machinery, vehicles, and physical plants that are used to process the mail. Because of the huge volume of mail sent daily, a piece by piece mail treatment would not be economically feasible; however, a system that treats all mail in bulk would assist in assuring a measure of safety to users of the mail system.

While it has been proposed to irradiate mail with gamma radiation (from radioactive cobalt) and with energetic electron beams, such irradiation systems can damage electronic devices (particularly memory chips) and expose photographic film being shipped in the mails. Also, such irradiation damages paper mail, discoloring it and making it brittle. It may result in degradation leading to the release of toxic vapors.

Radiation destroys or damages, or causes software errors in virtually all electronics including smart credit cards, all electronic devices including mobile phones, stereophonic equipment, cameras, and the like and damages or exposes film, and yellows photographic prints and also paper. Also, the equipment used to irradiate mail is very expensive and lead times are long. Such equipment has expensive components that wear out, and maintenance costs are high. If mail must be treated one piece at a time, many such expensive machines must be purchased to handle large volumes of mail.

U.S. Pat. No. 4,764,351 discloses a sterilization method and apparatus using a gaseous agent for sterilizing a gas for use in treating materials. U.S. Pat. No. 5,322,603 discloses a method of an apparatus for treating infections medical wastes is which large sizes of medical waste in a sealed body are exposed to microwaves and heat.

U.S. Pat. No. 5,470,546 discloses apparatus for storing and sterilizing bio-hazardous waste in which air is evacuated and pressurized steam is injected.

U.S. Pat. No. 5,591,117 discloses a method and an apparatus for the disposal of material containing infective microorganisms such as bacteria, fungi and viruses by introducing the material into a container which can be charged with ozone and exposed to the action thereof until the microorganisms are killed, and then the ozone is discharged from the container and converted to a lower valence level and the container is then evacuated. This treatment is designed to decontaminate medical waste which is later thrown away and thus one need not have concern about damaging it, whereas one is concerned about damaging mail.

U.S. Pat. No. 5,700,426 discloses a method for decontaminating or sterilizing “in situ” a vacuum sealed container and device for implementing such method for sterilizing or decontaminating microorganisms or dangerous products.

U.S. Pat. No. 6,159,422 discloses methods and apparatus for the treatment of hazardous biological waste materials. A biological waste material is placed into a chamber and a vacuum applied. Water vapor is into the chamber and electromagnetic radiation energy is applied to produce a plasma.

### SUMMARY OF THE INVENTION

In view of the above, the present invention provides a system and method for decontaminating and/or sanitizing mail using a gas which degrades or deactivates bio-hazardous materials, including anthrax spores. An example of such a gas is chlorine dioxide (ClO₂), which degrades or deactivates bio-hazardous materials such as anthrax. A quantity of mail is placed in a sealable chamber and subject to a vacuum pumping process by which the pressure in the chamber is reduced to a sub-atmospheric level, for example, 0.5 atm. Thereafter, the sub-atmospheric pressure chamber is filled with chlorine dioxide to some pressure, e.g.., atmospheric, to completely fill the interior interstices of the mail being treated to kill any disease-bearing bacteria or spores. After a sufficient period of time, the active gas is removed and the chamber back-filled with ambient air. If need be, the last step, or the entire process, can be repeated.

The present invention advantageously provides a method for decontaminating and/or sanitizing mail utilizing an active gas that eliminates the disadvantages associated with irradiation by energetic particles.

Other features and advantages will be apparent from the following detailed description of preferred embodiments taken in conjunction with the accompanying drawings.

### DESCRIPTION OF THE DRAWINGS

FIG. 1 is a schematic diagram of the system of the present invention.

FIG. 2 is a schematic block diagram of the processing steps of the present invention.

FIG. 3 is a schematic isometric view of mail trays on a cart in a processing chamber.

### DESCRIPTION OF THE PREFERRED EMBODIMENTS OF THE INVENTION

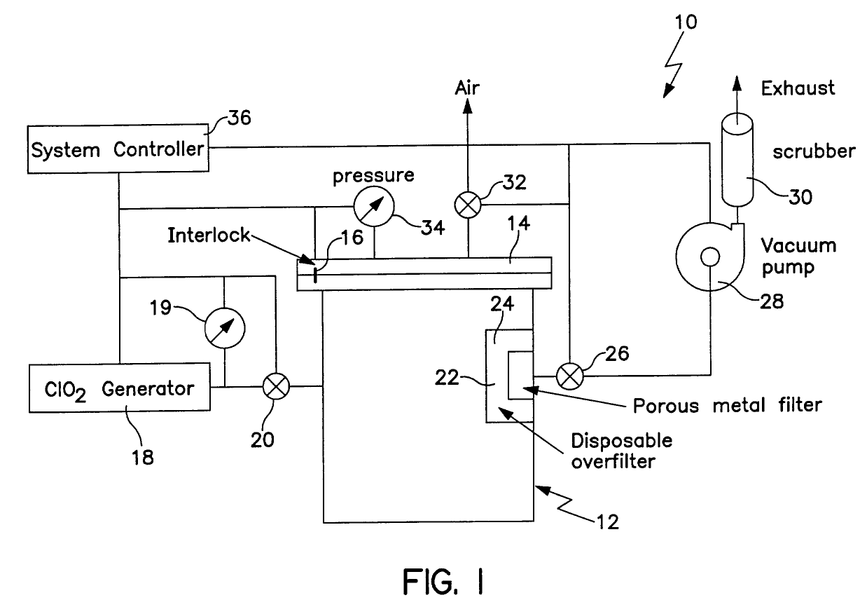

A system for decontaminating and/or sanitizing mail in accordance with the present invention is shown in FIG. 1 and designated generally therein by the reference character 10. As shown, the system 10 includes sealable chamber 12 designed to accept mail in a selected quantity, i.e., bulk mail. The chamber 12 includes a lid or cover 14 that can be opened and closed as desired; the cover 14, when closed, forms a gas-tight closure. An interlock 16 can be provided as part of the chamber 12 structure.

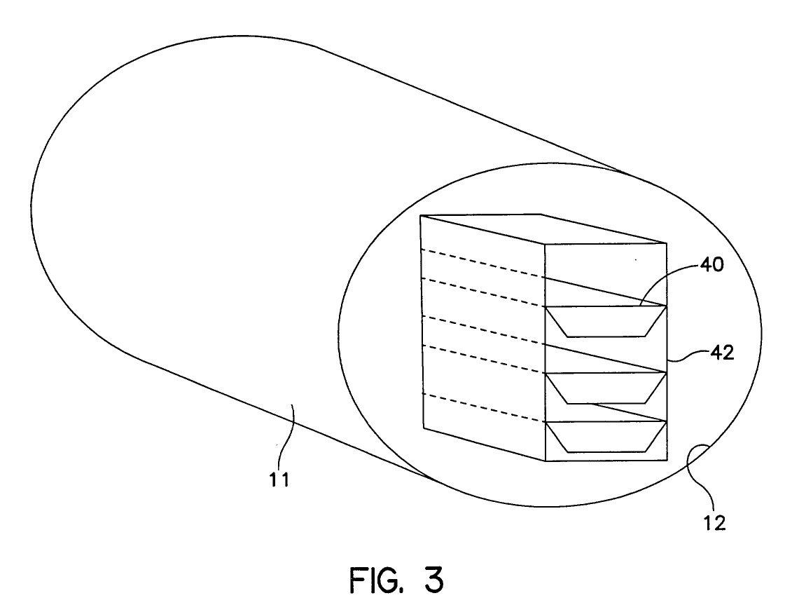

As an alternative, an autoclave 11 may be used which has a processing chamber 12 as shown in FIG. 3. In this event, the mail may be placed into trays or tubs 40, a number of which are placed onto wheeled carts 42 (only one is shown) which are then wheeled into the chamber 12 for processing.

A gas generator 18 having a pressure gauge 19 is provided to generate or otherwise supply a quantity of gas that degrades or deactivates anticipated biohazards. In the preferred embodiment, chlorine dioxide (ClO₂) is the preferred gas. The gas generator 18 is connected to the chamber 12 via a controllable valve 20. The gas generator also controls the water vapor content of the active gas.

The chamber 12 is selectively exhausted to the atmosphere via first filter 22 and a second filter 24 through a selectively controlled valve 26. The valve 26 is connected to a scavenge vacuum pump 28 and to a scrubber 30 that removes the chlorine dioxide gas prior to being exhausted to the ambient air.

Additionally, a selectively controllable valve 32 can be controlled to admit ambient air into the chamber 12, and a pressure gauge 34 is provided to indicate pressure in the chamber 12.

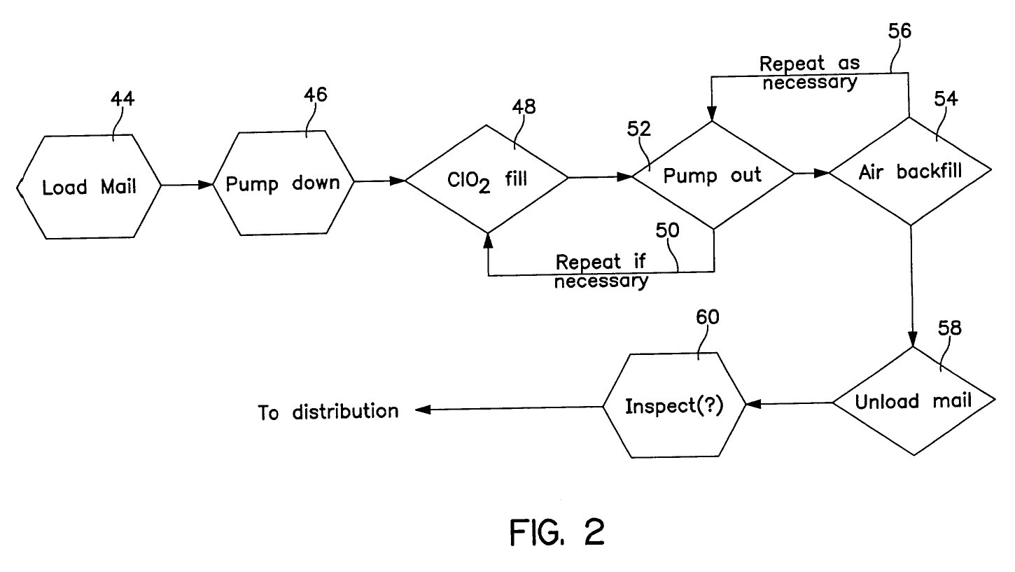

A system controller 36, in the form, for example, of a programmed microprocessor, computer, or functionally equivalent device, is connected to the various components to implement the method described below in FIG. 2.

The method of the present invention is shown in FIG. 2 and is representative of techniques for exposing the mail to the active gas. More specifically, the to-be-treated mail is loaded into the chamber 12 in step 44, Load Mail, and, after the chamber 12 is sealed close, valve 26 is opened, and the vacuum pump 28 is operated to provide step 46, Pump Down, the 12 to a sub-atmospheric pressure. In general, a pressure of about 0.5 atm is sufficient.

After the chamber 12 is pumped down, the valve 26 is closed and the valve 20 is opened to admit the chlorine dioxide into the chamber 12 to provide step 48, ClO₂ Fill. In general, the pressure of the chlorine dioxide can be somewhat below atmospheric pressure, at atmospheric pressure, or above atmospheric pressure. Since the chamber 12 was initially at a lower pressure, the chlorine dioxide will flow into, or otherwise diffuse into, the interior of each piece of mail and into interstices in the interior of the mail. The gas will enter, for example, by diffusion through the envelope walls or through less-than fully sealed seams, flaps and the like. In general, a concentration of about 4% or less chlorine dioxide is adequate.

The mail is exposed to the gas for a selected period of time empirically determined to deactivate or degrade and render harmless the target bio-hazardous material. If it is determined that the bio-hazardous material has not completely rendered the bio-hazardous material harmless, step 50, Repeat As Necessary, is performed and a further treatment with the gas is performed. This can be accomplished in different manners depending upon the circumstances. For example, if the material is not killed or rendered harmless, the mail can remain in the gas for a longer period of time. If desired, a gas flow can be provided, such as by a pump or fan to circulate the gas throughout the chamber 12 including through the mail containers until the bio-hazardous material has been neutralized or decontaminated. In some it may be prudent to go to step 52, Pump Out, first and then go to steps 50 and 48 in which the chamber will be refilled with fresh active gas.

Thereafter, the valve is 26 is opened to allow the vacuum pump 28 to scavenge the gas from the chamber 12 while the valve 32 is opened thereafter to allow ambient air to enter the chamber 12 and effectively purge the chamber 12.

As shown, the process of steps 52, Pump Out, and 54, Air Backfill, can be repeated in step 56, Repeat As Necessary, fill can be repeated as necessary. Sensors for chlorine dioxide, chlorine, or for other active gases and their degradation products, can be attached to the chamber 12 to determine whether or not the chamber has been purged sufficiently to e safely opened. When the gas has been purged to the necessary extent, step 58, Unload Mail, is performed, and, if desired, step 60, Inspection, can be done and the mail then continues to distribution.

The present invention advantageously provides a method for decontaminating/sanitizing mail that efficiently exposes both the exterior and the interior of the mail pieces, and the interstices therein, to a gas that degrades or deactivates, and renders harmless, targeted biohazards.

Thus, as described the present invention is a method (and the apparatus) for use of chlorine dioxide (ClO₂) or another substance to kill biological warfare agents held on or inside mail. The method can be applied using virtually any other type gas that deactivates or degrades and renders biological warfare agents, including ethylene oxide, other chlorine containing species and others. However, the present disclosure describes the use of ClO₂ as one preferred embodiment because it has been shown to be effective against biological agents, such as anthrax spores.

In the present invention, mail, (either as individual pieces or as items in trays, held in baskets or bins, which are in turn placed onto wheeled racks, or transported by automated means or fork lifts, or any other method of holding and transporting batches of mail in such a way that each piece is in contact with air), is wheeled into a vacuum chamber.

This can be an autoclave, such as those that are used for processing composite structures like aircraft and satellite components, and the like, or hyperbaric chambers, or other vacuum chambers, and a vacuum is created sufficient to remove air from around and inside the letters or packages. A “hard” vacuum is not required in such an application. A vacuum of 1/2 atmosphere (pressure 389 torr) or even 9/10 atmosphere (700 torr) would be suitable for this application. The chamber is then backfilled with a ClO₂/air mixture generated by a commercial ClO₂ generator such as that made by CDG Technology, or generators made by CDG's competitors. Controlling the humidity of the active gas is important. Because a partial vacuum exists inside the chamber, and also inside the letters and parcels in the chamber, gas will surround and fill the letters when the chamber is filled with the ClO₂/air mixture. It has been shown that 100 ppm of ClO₂ in air, held for 4 hours, will kill every single spore held on a test strip that contains 1,000,000 Bacillus var. Niger spores (also known as Bacillus Globigii or simply “BG”). BG is a spore widely used as a simulant for the nearly identical Bacillus anthracis spores that are responsible for anthrax disease. Higher concentrations of ClO₂ in air, which could be 1,000 ppm or even a little higher, will act faster to reduce the concentration of remaining viable spores to zero, or to a very small number that will be insufficient to cause cases of either inhalation anthrax (requiring 8,000 to 50,000 spores to be breathed in for infection to occur) or cutaneous anthrax in most cases. For such spores, proper control of humidity helps the gas permeate the spore coat and degrade or deactivate the spores, rendering them harmless.

After this treatment of the mail is complete, the chamber is again evacuated to the previous level and is refilled with air. This process may need to be repeated at least 3 times, or more as needed, to remove residual ClO₂ gases that might otherwise remain in the letters or packages. Exhaust ClO₂/air mixtures, or air contaminated with small amounts of ClO₂ are passed through a simple water scrubber made of polyvinyl chloride plastic (PVC) or other materials to remove all ClO₂ before it is exhausted into the air. The scrubber liquid can usually be safely discharged into a sewer system, since no biohazards or ClO₂ will remain. An environmental health and safety specialist would usually be consulted, however, on applicable regulations relating to the discharge and handling of this scrubber solution.

After the multiple evacuation/air refill cycles are finished, residual ClO₂ levels will be low. They can be detected in real time inside the chamber it has been refilled with air. Sensors for ClO₂, or sensors for Cl, will detect the presence of residual chemical to quite low levels. Commercially available electrochemical, solid state (metal oxide sensors) and spectroscopic sensors can be used. After this evacuation/refill process is finished, the mail is removed from the chamber and processing continues.

The ClO₂ concentration vs. decontamination time relationship can be calibrated for different sized mail pieces, and for bundled bulk mail, so that each can be treated in an optimal method, or so that a single method applicable to the “worst case” condition, can be used. This process is a batch process taking place before mail enters a main mail processing and distribution center, a company's mail room, and the like. For high volumes of mail, multiple chambers may be required, although they could be served by the same pumping system and the same ClO₂ generator. Mail normally waits for quite some time to enter the processing and distribution center, so that little or no additional time is added to the overall mail processing and distribution sequence. This system can also be used by small company mail rooms that will treat just one or two batches of mail per day and will thus only need one chamber.

Any small residual chlorine, the breakdown product of ClO₂, or residual ClO₂ itself, will be at such low levels that they will not be harmful to people. They will be detectable to mail recipients as a “swimming pool”-like odor. This will likely dissipate during the time that elapses between mail decontamination and receipt of the mail by the public. ClO₂/air mixtures are simply made from chlorine gas (Cl) brought in cylinders, and sodium chlorite (NaClO₂), a material that can be contained in 55 gallon drums, and a humidified air stream. They can be made in various concentrations of ClO₂ in air, but are best held below about 1% ClO₂ in air, as the explosive limit for this gas is in the 4-10% range. For this reason, ClO₂ is never shipped, but is always produced where and when it is required. Thus, the nature of the generation equipment and the ClO₂ generation process make it impossible to exceed the explosive limit, so an explosion hazard will not exist. The same type of standard, commercial ClO₂ generators and technology used for various commercial and hospital biological purification applications, will be used to make the ClO₂/air decontaminant mixture for mail and parcel purification.

As will be apparent to those skilled in the art, various changes and modifications may be made to the illustrated method for decontaminating and/or sanitizing mail of the present invention without departing from the spirit and scope of the invention as determined in the appended claims and their legal equivalent.

1. A method of de-contaminating and/or sanitizing mail comprising the steps of:
a. placing a quantity of mail into a confined chamber;
b. evacuating the atmosphere in the chamber to a sub-atmospheric pressure;
c. filling the chamber with an active gas that degrades or deactivates targeted bio-hazardous materials, which step is assisted by the sub-atmospheric pressure in the chamber provided in step b.; and
d. purging the active gas from the confined chamber.

2. A method as defined in claim 1 wherein steps c. and d. are repeated as needed.

3. A method as defined in claim 1 wherein the bio-hazardous material is anthrax spores and the active gas is ClO₂.

4. A method as defined in claim 3 wherein the humidity is controlled.

5. A method as defined in claim 1 wherein the chamber is sufficiently large to contain a large plurality of mail bins.

6. A method as defined in claim 4 wherein the mail bins are removably placed onto wheeled carts which are wheeled into the chamber prior to the step of evacuating the atmosphere.

7. A method as defined in claim 1 wherein the sub-atmospheric pressure is between 0.5 and 0.9 atm.

8. A method as defined in claim 1 wherein the active gas introduced is at least 100 ppm in air and the gas is maintained in the chamber for four hours.

9. A method as defined in claim 1 wherein the active gas introduced is 1000 ppm in air and the gas is maintained in the chamber for substantially less than four hours.

10. A method as defined in claim 1 wherein the step of purging is continued until the active gas is reduced in concentration to a safe level.

11. A method of de-contaminating and/or sanitizing mail comprising the steps of:
a. loading mail into a confined chamber;
b. partially evacuating the atmosphere in the chamber;
c. filling the chamber with an active gas that deactivates or degrades, and renders harmless one or more targeted bio-hazardous materials;
d. maintaining the mail and gas in the chamber for a sufficient period of time to neutralize the bio-hazardous material;
e. pumping the gas out of the chamber and allowing air to enter to reduce the active gas to a safe level;
f. examining the chamber to determine whether the bio-hazardous material has been neutralized;
g. if the bio-hazardous material has not been neutralized, repeating steps b. through f.;
h. in the event the bio-hazardous material has been neutralized, examining the chamber to determine whether the active gas has been reduced to a safe level;
i. in the event, the active gas is not at a safe level, repeating steps b. through h.; and
j. in the event the active gas is at a safe level, unloading the mail from the chamber.

12. A method as defined in claim 11, wherein steps b. through i. are carried out under the control of a computer.

13. A method as defined in claim 11, wherein said chamber is sufficiently large to contain a large plurality of mail bins and the step of loading mail includes loading a large plurality of mail bins.

14. A method as defined in claim 13, wherein said chamber is sufficiently large to contain a plurality of wheeled carts each of which can support a plurality of mail bins and the step of loading mail includes loading a plurality of wheeled carts.

15. A system for de-contaminating and/or sanitizing mail, comprising:
a. a container having a confined chamber;
b. means for evacuating the atmosphere in the chamber to a subatmospheric pressure;
c. means for filling the chamber with a gas that deactivates or degrades, and renders harmless, one or more targeted bio-hazardous materials; and
d. mains for purging the active gas from the confined chamber.

16. A system as defined in claim 15, further comprising a system controller for controlling the means for evacuating, means for filling and means for purging the de-contaminating and/or sanitizing mail.

17. A system as defined in claim 15, wherein said container is sufficiently large to contain a large plurality of mail bins.

18. A system as defined in claim 17, wherein said container is sufficiently large to contain a plurality of wheeled carts each of which can support a plurality of mail bins.
